(19) United States
(12) Patent Application Publication
(10) Pub. No.: US 2009/0216096 A1
(43) Pub. Date: Aug. 27, 2009

(54) METHOD AND APPARATUS TO DETERMINE SKIN STEROL LEVELS

(75) Inventors: Matthew B. Bloom, San Francisco, CA (US); Dana L. Deardorff, Lafayette, CO (US); Gilbert Hausmann, Felton, CA (US); Paul D. Mannheimer, Danville, CA (US); Michael P. O'Neil, Pleasanton, CA (US); Shannon E. Campbell, Boulder, CO (US); Rafael Ostrowski, Pittsburg, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/345,377

(22) Filed: Dec. 29, 2008

Related U.S. Application Data
(60) Provisional application No. 61/009,681, filed on Dec. 31, 2007.

(51) Int. Cl. A61B 5/1455

(52) U.S. Cl. 600/322

(57) ABSTRACT

A method and apparatus is provided for measuring a sterol, such as cholesterol, deposited in the skin. Light of two or more wavelengths is emitted toward or into a skin sample and the light is differentially absorbed by the sterol in the skin sample. The differentially absorbed light is detected and the amount of sterol in the skin sample is determined based on the differential absorption.

### RELATED APPLICATION

This Application claims priority from U.S. Pat. Application No. 61/009,681 which was filed on Dec. 31, 2007, and is incorporated herein by reference in its entirety.

### BACKGROUND

The present disclosure relates generally to the measurement of tissue constituents. In particular, the disclosure relates to the measurement of cholesterol in the body.

This section is intended to introduce the reader to various aspects of art that may be related to various aspects of disclosed embodiments, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Cholesterol is a needed component of various structures in the body, such as cellular membranes. However, in excess amounts or in other adverse circumstances cholesterol can pose a health problem or can serve as a predictor of health problems. For example, elevated cholesterol frequently give rise to xanthomas, deposits of cholesterol in peripheral tissues and may also result in cholesterol being deposited or “plating out” of the blood to form plaques in arterial blood vessels, thereby restricting blood flow and leading to coronary and circulatory disorders. Indeed, such atherosclerotic disease is a leading cause of death in western countries. It is, therefore, desirable to identify individuals who are prone, for environmental and/or genetic reasons, to atherosclerosis so that preventative measures and risk reduction strategies may be implemented as early as possible.

Such high-risk individuals are typically identified by measuring the cholesterol levels in their blood serum. A blood serum measure of cholesterol, however, is heavily influenced by liver function, and therefore does not directly assess the extent of cholesterol deposition in the tissues of the patient. Therefore, a serum cholesterol measurement level is, at best, merely a surrogate measure of cholesterol deposition in the tissues.

Further, techniques that measure serum cholesterol are typically invasive in nature and require the extraction of a blood sample. Such techniques may be both physically and psychologically uncomfortable for a patient. As a result of these discomforts, a patient who would benefit from frequent, or at least periodic, screening, may forego such screening, thus making early detection of atherosclerotic disease less likely.

It may, therefore, be desirable, to provide for a more direct measure of cholesterol deposition in the tissues of a patient. Likewise, it may be desirable to provide for the non-invasive measurement of cholesterol deposition.

### SUMMARY

Certain aspects commensurate in scope with this disclosure are set forth below. It should be understood that these aspects are presented merely to provide the reader with a brief summary of certain forms of the invention might take and that these aspects are not intended to limit the scope of the invention. Indeed, the invention may encompass a variety of aspects that may not be set forth below.

According to an embodiment, there may be provided a method for measuring accumulated cholesterol in tissue. The method includes the act of emitting light of two or more wavelengths toward a skin sample such that the light is differentially absorbed by plated or platable cholesterol in the skin sample. The differentially absorbed light is detected. The amount of plated or platable cholesterol in the skin sample is determined based on the differential absorption.

According to an embodiment, there may be provided a system for measuring accumulated cholesterol in tissue. The system includes one or more light emitters. The one or more light emitters are each configured to emit light at one or more wavelengths such that the combined one or more light emitters emit light at two or more wavelengths. The system also includes one or more light detectors configured to detect the light. The system also includes a processor configured to receive the detected light and to determine an amount of plated or platable cholesterol in the skin based on differential absorption of the light.

According to an embodiment, there may be provided one or more computer-readable media on which a computer program is embodied. The computer program includes a routine configured to receive one or more signals representing the differential absorption of light by plated or platable cholesterol in a skin sample. The media also include a routine configured to generate a measure of the plated or platable cholesterol based upon the one or more signals.

According to an embodiment, there may be provided a system for measuring a sterol. The system includes one or more light emitters each configured to emit light at one or more wavelengths such that the combined one or more light emitters emit light at two or more wavelengths comprising at least one wavelength between about 1,462 nm to about 1,482 nm, between about 1,703 nm to about 1,723 nm, between about 1,740 nm to about 1,760 nm, between about 2,066 nm to about 2,086 nm, or between about 2,300 nm to about 2,320 nm. The system also includes one or more light detectors configured to detect the light. In addition) the system includes a processor configured to receive the detected light and to determine an amount of a sterol deposited in the skin based on differential absorption of the light.

According to an embodiment, there may be provided a system for measuring a sterol deposited in the skin. The system includes one or more light emitters each configured to emit light at one or more wavelengths such that the combined one or more light emitters emit light at two or more wavelengths. The system also includes one or more light detectors configured to detect the light and a processor configured to receive the detected light and to determine an amount of a sterol deposited in the skin based on differential absorption of the light. In addition, the system also includes one or more optically transmissive structures in optical communication with at least some of the one or more light emitters and/or the one or more light detectors. The one or more optically transmissive structures are configured to penetrate the surface of an in vivo skin sample.

### BRIEF DESCRIPTION OF THE DRAWINGS

Advantages of this disclosure may become apparent upon reading the following detailed description and upon reference to the drawings in which:

FIG. 1 illustrates a block diagram of a diagnostic system in accordance with aspects of an exemplary embodiment;

FIG. 2 illustrates a block diagram of a diagnostic system in accordance with aspects of a further exemplary embodiment;

FIG. 3 illustrates a block diagram of a diagnostic system in accordance with aspects of an additional exemplary embodiment; and

FIG. 4 is a flowchart depicting exemplary acts for measuring the amount of a sterol deposited in a tissue in accordance with aspects of an exemplary embodiment.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present technique is generally directed to the measurement of sterols, such as cholesterol, using spectrophotometric techniques. For example, the amount of cholesterol deposited in the skin or other externally accessible tissues may be measured and used in determining patient risk for diseases associated with cholesterol deposition, such as atherosclerosis and other ischemic diseases. Indeed, skin cholesterol levels are believed to be better correlated with coronary atherosclerosis disease and other coronary disorders than the measures of serum cholesterol currently employed. In particular, cholesterol is believed to be deposited in the skin in the same manner and at similar rates as it is deposited in other tissues, such as arterial tissues. Therefore, the amount of cholesterol deposited in the skin is believed to be a good indicator of the amount of cholesterol deposited in other tissues, such as vascular tissues.

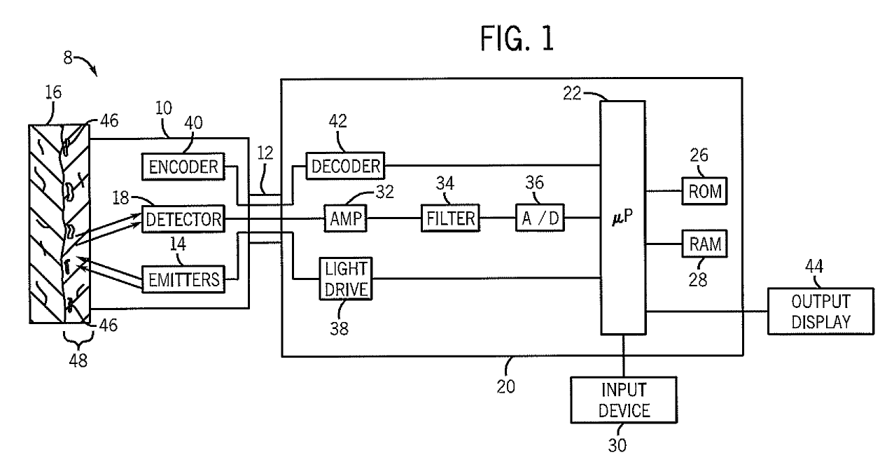

The use of spectrophotometric devices to detect sterols, such as cholesterol, may provide advantages such as early detection and ease of use. Additionally, spectrophotometric devices are non-invasive. FIG. 1 illustrates a block diagram implementing a spectrophotometric device in a diagnostic system 8 in accordance with an exemplary embodiment of the present technique. The diagnostic system 8 includes a sensor 10 having an emitter or emitters 14 configured to transmit electromagnetic radiation, such as light, into a skin sample 16 of a patient.

In an embodiment where the diagnostic system 8 is configured to measure skin cholesterol, the emitters 14 may be configured to emit light at wavelengths that are differentially absorbed by cholesterol, such as cholesterol deposited in the skin sample 16. In one such an embodiment, the emitters 14 emit at least two wavelengths of light, either concurrently or alternately, where the two wavelengths are differentially absorbed by deposited cholesterol. For example, in such an embodiment, at least one of the two wavelengths may be in the range of about 1,200 nm to about 2,400 nm. Exemplary ranges in such an embodiment are between about 1,462 nm to about 1,482 nm (such as about 1,472 nm), between about 1,703 nm to about 1,723 nm (such as about 1,713 nm), between about 1,740 nm to about 1,760 nm (such as about 1,750 nm), between about 2,066 nm to about 2,086 nm (such as about 2,076 nm), or between about 2,300 nm to about 2,320 nm (such as about 2,310 nm).

The emitter or emitters 14 may be provided as separate and discrete devices, such as in an implementation where a discrete emission device is provided for each wavelength of light to be emitted. An example of such emission devices may be separate and discrete light emitting diodes (LEDs) for each wavelength of interest. Alternately, for wavelengths where an LED may be unavailable or undesirable, an optical engine or other device capable of generating the desired wavelength of light may be provided, either in the sensor 10 or spectrophotometric device 20. In such an embodiment, the transmission of the light to and through the sensor 10 to the skin sample may be accomplished via fiber optics or other optically transmissive media. In an additional embodiment, the emitter 14 may be provided as a single “white light” source, i.e., a source that emits light across a narrow or broad spectrum of wavelengths. In such an embodiment, the spectrum of wavelengths at which the emitter 14 emits includes the two or more wavelengths of interest.

In one embodiment, the sensor 10 is placed on the patient's skin and is removed from the patient after the examination is concluded. In such an embodiment, the electromagnetic radiation is scattered and absorbed by the various constituents of the patient's tissues, such as skin cholesterol. In the depicted embodiment, a photoelectric detector 18 in the sensor 10 is configured to detect the scattered and reflected light and to generate a corresponding electrical signal. Examples of such a photoelectric detector 18 include a photodiode configured to detect light at one or more of the wavelengths of interest. The number and type of photoelectric detectors 18 will typically depend on the number and type of emitters 14 and/or on the light emission scheme employed. For example, in an embodiment in which emitters 14 only emit light at the wavelengths of interest and in which the emissions alternate, i.e., only light at one wavelength is emitted, a single detector 18 may be provided if the detector 18 is configured to detect light at each wavelength of interest. Alternately, a detector 18 may be provided for each wavelength of interest, such as in embodiments where light at more than one wavelength of interest is concurrently emitted. Likewise, a wavelength discriminating detector 18 may be employed where light at more than one wavelength of interest is concurrently emitted or where a “white light” type source is employed which emits light at not only the wavelengths of interest but at other wavelengths as well.

In an exemplary embodiment, the sensor 10 directs the detected signal from the detector 18 into a spectrophotometric device 20, such as via cable 12. In this embodiment, the spectrophotometric device 20 may have a microprocessor 22 that calculates sterol levels, such as cholesterol levels, using algorithms programmed into the spectrophotometric device 20. In such an embodiment, the microprocessor 22 may be connected to other component parts of the spectrophotometric device 20, such as a ROM 26, a RAM 28, and input device(s) 30. In one embodiment, the ROM 26 holds the algorithms used to compute the sterol levels and the RAM 28 stores the values detected by the detector 18 for use in the algorithms.

In one embodiment, input device 30 allows a user to interface with the spectrophotometric monitor 20, such as via buttons of an operator interface, a keypad or keyboard, or a mouse or other selection mechanism for use with a provided control interface. For example, a user may input or select parameters specific to the patient or skin sample 16 undergoing measurement or may specify a measurement protocol where multiple protocols are available. For example, different wavelengths or wavelength combinations and/or different light emission timing schemes or measurement cycle lengths may be utilized in different protocols. As a result, different protocols may be desirable under different patient or skin sample circumstances or to provide secondary or independent sterol measurements. Additionally, patient data may be entered, such as sex, weight, age, body mass index, and medical history data, including, for example, whether a patient suffers from obesity, diabetes, heart disease, metabolic disorders, auto-immune pathologies, and so forth. This information may be used to validate the baseline measurements or to assist in the understanding of anomalous readings. For example, the age, sex and/or weight of a patient may alter the baseline reading of skin cholesterol and, therefore, would affect any determination of clinical or medical significance in the reported measure or score.

Detected signals are passed from the sensor 10 to the spectrophotometric device 20 for processing. In the depicted embodiment, the signals are amplified and filtered in the spectrophotometric device 20 by amplifier 32 and filter 34, respectively, before being converted to digital signals by an analog-to-digital converter 36. The signals may then be used to determine the sterol measure and/or stored in RAM 28.

In one embodiment, a light drive unit 38 in the spectrophotometric device 20 controls the timing of the emitter or emitters 14. While the emitters 14 are manufactured to operate at one or more certain wavelengths, variances in the wavelengths actually emitted may occur which may result in inaccurate readings. To help avoid inaccurate readings, an encoder 40 and decoder 42 may be used to calibrate the spectrophotometric monitor 20 to the actual wavelengths being used. The encoder 40 may be a resistor, for example, whose value corresponds to coefficients stored in the spectrophotometric device 20. The coefficients may then be used in the algorithms. Alternatively, the encoder 40 may also be a memory device, such as an EPROM, that stores information, such as the coefficients themselves. Once the coefficients are determined by the spectrophotometric device 20, they are inserted into the algorithms in order to calibrate the diagnostic system 8. Though the encoder 40 is depicted in the sensor 10 in FIG. 1, the encoder may, alternatively, be provided in the cable 12 in embodiments in which the sensor 10 and cable 12 are not separable.

The spectrophotometric device 20 may be configured to display the calculated parameters, such as the measured sterol, on display 44. As the sterol measurement may not have any particular significance to a caregiver or clinician, the spectrophotometric monitor may be programmed to correlate this measurement to a number or other indicator corresponding to a more readily understood measure of a risk level or of a potential stage of development of a particular condition. For example, sterol levels may be provided as percentage, quartile, or decile readings that may be more intuitively understood by a clinician or caregiver. Such indicators, along with or instead of the actual sterol measurement, may be outputted on the display 44. In some embodiments, a color display may also be programmed to correlate the sterol readings with a particular color. For example, a green, yellow or red light may be shown on the display corresponding to normal, abnormal, and severely abnormal readings, respectively. The color may be used independently or in combination with a numerical or graphical indicator scheme. Regardless of the manner of presentation, the objective is to present the sterol information to a clinician in a manner that may be quickly and easily understood. For example, the results may be graphically presented (such as using bar-, line-, or pie-charts or other types of graphical presentation tools) to provide context and comparison to an evaluating clinician, thereby allowing the clinician to make more informed judgments.

With regard to the tissue undergoing examination, the skin sample 16 examined by the sensor 10 may be in vivo, i.e., still on the patient. For example, the skin sample 16 may be the skin of the heel or palm of a patient, such as the skin of the thenar plexus. In such implementations, the sensor 10 may be provided as a reflectance-type spectrophotometric sensor in which the emitters 14 and detectors 18 are on the same side of the skin sample 16, as generally schematically represented in FIG. 1. Such sensor implementations may be suitable for measurement of absolute quantities of sterols, such as cholesterol 46, which are plated or are susceptible to plating in the stratum corneum 48 (or in the epithelial layers in general) as well as in mucosal tissues or in optic tissues, such as the retina.

In some implementations where the skin sample 16 is in vivo, light emitted by the emitters 14 penetrates less than about 3 mm. For example, in one embodiment, the light emitted by the emitters 14 penetrates about the upper 200 μm of the skin. Likewise, in some embodiments, the light emitted by the emitters 14 penetrates into the stratum corneum 48, which may be 50 μm to 100 μm in depth, depending on the location of the skin sample 16, but not substantially beyond the stratum corneum. In such an embodiment, the emitted light may be useful for assessing the amount of cholesterol 46 plated or susceptible to plating throughout the stratum corneum, including at the bottom of the stratum corneum.

In some embodiments the wavelengths and intensity of the emitted light may be selected so as not to penetrate as deep as the lipid layer underlying the skin. However, in implementations where the light emitted by the emitters 14 may penetrate to the lipid layer, the effect of the lipids on the emitted light may be separated out to allow accurate measurement of the sterol deposited in the skin. For example, in implementations where lipid effects on the emitted light are possible, such as due to differential absorption of the emitted light by the lipid layer, the emitters 14 and detector 18 may be provided on the sensor 10 in paired arrangements or as an array having different distances between combinations of emitters 14 and detectors 18. Such an array or paired arrangements would allow depth resolution of the detected signals. In such an arrangement, there may be actual pairs of discrete emitter and detector locations at different distances or there may be a single emitter location in combination with multiple detectors at different distances from the emitter. Conversely, a single detector location may be provided in combination with multiple emitter locations at different distances from the detector.

In such implementations, the light activation scheme may be such that only a given emitter-detector pair is active at one time so that for each distance between an emitter 14 and a detector(s) 18 there is a discrete measurement signal. The signals corresponding to the greater distances between the emitters 14 and the detector 18 will include whatever lipid effects, if any, are present and can be used to estimate light absorption effects attributable to the lipid layer. This estimate may in turn be used to remove the lipid layer effects to provide an accurate measurement of sterol deposited in the skin sample.

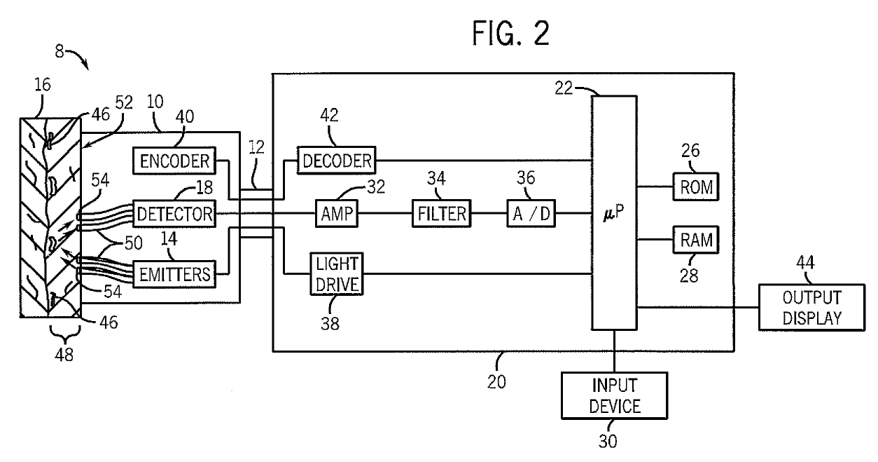

The embodiment of FIG. 1, generally depicts the use of spectrophotometric techniques to measure sterols, such as cholesterol, plated or susceptible to plating in an in vivo skin sample. A further embodiment of these techniques is depicted in FIG. 2, in which the emission and/or detection mechanisms penetrate the surface of the in vivo skin sample 16. In particular, in the embodiment depicted in FIG. 2, each emitter 14 and/or each detector 18 are coupled to one end of respective optically transmissive structures, such as fiber optic strands 50. The other end of the optically transmissive structures penetrates the surface 52 of the skin sample 16 such that the tips 54 of the optically transmissive structures are disposed within the stratum corneum 48, but do not generally pass completely through the stratum corneum 48. For example, in such an embodiment, the tips 54 may penetrate about 25 μm to about 100 μm beneath the surface 52 of the skin sample 16. In this manner, the light or other electromagnetic radiation is emitted and/or detected directly in the stratum corneum 48.

In one embodiment, the tips 54 may be the tips of optically transmissive microneedles, such as metalized microneedles having an optically transmissive core. Examples of the manufacture and use of such optically transmissive microneedles may be found in U.S. patent application Ser. No. 11/716,145, titled “SYSTEM AND METHODS FOR OPTICAL SENSING AND DRUG DELIVERY USING MICRONEEDLES” by Carine Hoarau et alt, filed on Mar. 9, 2007, herein incorporated by reference in its entirety. While optically transmissive microneedles are one example of an optically transmissive structure suitable for penetration of the surface 52 of the skin sample 16, other optically transmissive structures may also be employed. For example, the optically transmissive structure may be provided as tines, blades, as a sharpened end of a bare or coated fiber optic strand or bundle, or as any other suitable structure capable of penetrating the surface 52 of the skin sample 16 and transmitting the electromagnetic wavelengths of interest.

While the depicted example portrays both light emission and light detection using optically transmissive structures that penetrate the surface 52 of the skin sample 16, one of ordinary skill in the art will appreciate that other embodiments of the present technique may differ. For example, in one embodiment, light emission may be via optically transmissive structures penetrating the surface 52 of the skin sample 16 (as depicted in FIG. 2) while light detection may be via one or more detectors 18 disposed on or near the surface 52 of the skin sample 16 (as depicted in FIG. 1). Conversely, in another embodiment, light emission may be via one or more emitter 14 disposed on or near the surface 52 of the skin sample 16 (as depicted in FIG. 1) while light detection may be via optically transmissive structures penetrating the surface 52 of the skin sample 16 (as depicted in FIG. 2). Likewise, different depths of light emission and/or detection apparatuses may be employed such that some portion of light emission and/or detection occurs near or above the surface 52 of the skin sample, while the remainder of the emission and/or detection occurs beneath the surface 52 of the skin sample, such as via the optically transmissive structures discussed herein.

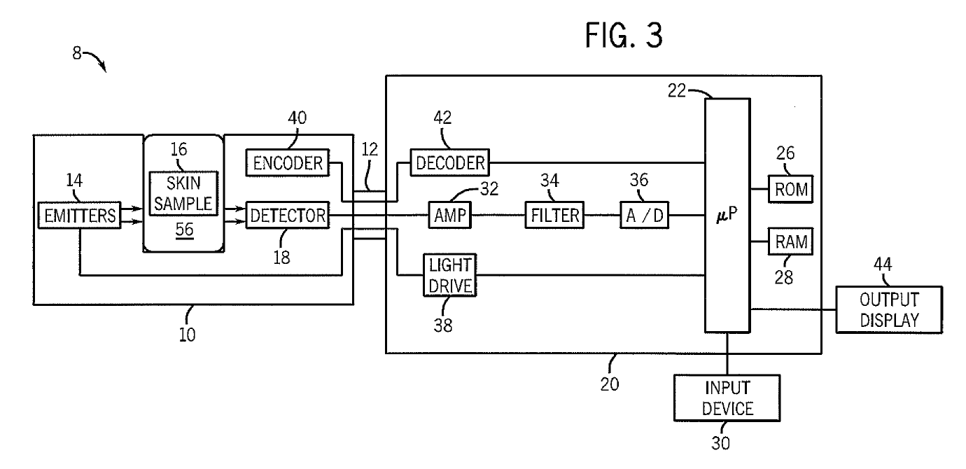

In other embodiments, the skin sample 16 may consist of skin cells that are removed from the patient, i.e., ex vivo, such as on an adhesive strip or substrate 56, as depicted in FIG. 3. For example, a tape or other adhesive substrate 56 may be applied to a patient's skin and removed, with some number of skin cells adhering to the adhesive substrate upon removal. The skin cells on the substrate may then be spectroscopically analyzed to assess levels of a deposited sterol in the skin sample. In such an embodiment, the sensor 10 may be provided in either a reflectance or transmission-type configuration, with a transmission-type configuration generally schematically illustrated in FIG. 3. In such a transmission-type configuration, the emitters 14 and detectors 18 are placed on opposing sides of the skin sample 16 such that the detectors 18 measure the amount of light of the wavelengths of interest that are transmitted through the skin sample 16. In such embodiments, therefore, the adhesive substrate 56 will typically be generally transparent to light at the wavelengths of interest.

In both FIGS. 1, 2 and 3, the light at the wavelengths of interest detected at the detector 18 is differentially absorbed by sterols, such as cholesterol, in the skin sample 16. This differential absorption of light at the wavelengths of interest may, therefore, be processed by microprocessor 22 to determine the amount of sterol in the skin sample 16, as described herein.

As will be appreciated, the skin sample 16, whether in vivo or ex vivo, may be treated with one or more chemical, biochemical, and/or enzymatic agents that may react with the sterol of interest, such as cholesterol. The reaction product may then be measured or assessed using spectrophotometry, as discussed herein, to determine an amount of cholesterol (or other sterol) present in the skin, such as in a plated form or in a form susceptible to plating. While some embodiments of the present technique may utilize such a reaction step, other embodiments measure the sterol in the skin directly, without the generation of a reaction product intermediary.

Likewise, in some embodiments, it may be desirable to apply a solvent to the skin of the patient to solubilize the sterol, thereby allowing the sterol to migrate within the skin, such as to the upper levels of the skin. For example, in an implementation where it is desired to use an adhesive substrate 56 to acquire an ex vivo skin sample 16, but where the sterol of interest is not believed to be generally deposited in the uppermost layers of skin cells (which are most susceptible to adhesion on the adhesive substrate 56), it may be desirable to solubilize the deposited sterol in deeper skin cells, i.e., those skin cells too deep to be adhered to the adhesive substrate 56. Once the plated or platable cholesterol (or other sterol) is solubilized, the solubilized sterol may migrate to skin cells that are more susceptible to adhesion, which may then be removed to form the skin sample 16 which undergoes analysis. In this manner, the skin sample 16 may be ex vivo and may include the deposited sterol of interest even though the sterol may typically be deposited in deeper tissue layers than those forming the skin sample 16.

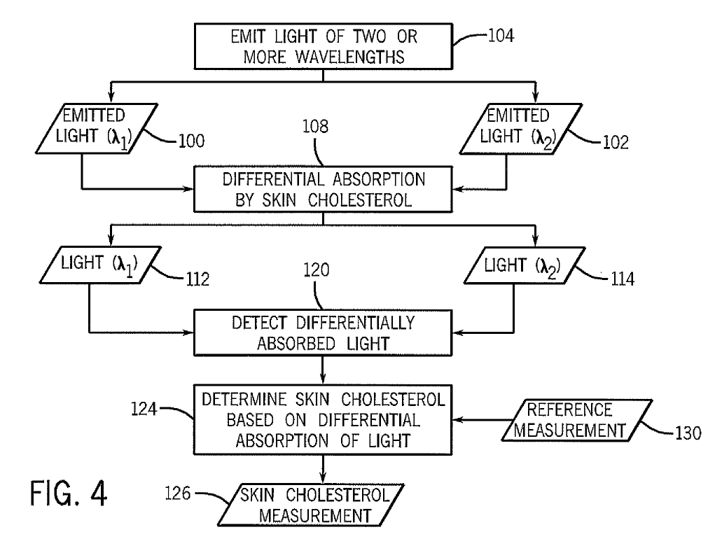

The preceding discussion generally describes devices suitable for measuring sterol levels, such as cholesterol levels, in a skin sample 16 using spectrophotometry. Referring now to FIG. 4, exemplary acts performed by or using devices such as those described in FIGS. 1, 2, and 3 are described. In an exemplary implementation, at least two wavelengths of light 100, 102 are emitted (block 104), such as by emitters 14 (FIGS. 1, 2 and 3). As previously noted, the two or more wavelengths of light 100, 102 may be emitted concurrently or in an alternating manner.

The emitted light 100, 102 of the two or more wavelengths is differentially absorbed (block 108) by sterols, such as plated or platable cholesterol, in a skin sample 16 (FIGS. 1, 2 and 3). In particular, light at one of the two or more wavelengths is absorbed to a different degree by the sterol deposits than light at the remaining wavelengths. As a result of the differential absorption, the relative amounts of the light 112 at the first wavelength and the light 114 at the second wavelength provide useful information regarding the quantity of sterol, such as cholesterol, plated or platable in the skin.

The differentially absorbed light 112, 114 is detected (block 120), such as with one or more detectors 18 (FIGS. 1, 2 and 3). In one embodiment, the detectors 18 may generate a signal for each wavelength of light, here two wavelengths, λ₁ and λ₂, such that each signal corresponds to the amount of light 112, 114 detected by the detector 18. In other embodiments, the detector 18 may generate a difference signal corresponding to the difference between the amounts of light 112, 114 at the two wavelengths that is detected by the detector 18.

Based on the signal or signals generated by the detector in response to the differentially absorbed light 112, 114, the amount of sterol present in the sample tissue, here skin cholesterol 126, is determined (block 124), such as by processor 22 (FIGS. 1, 2 and 3). For example, the processor 22 may execute one or more routines or algorithms for determining skin cholesterol based on the differentially absorbed light 112, 114. For example, in an exemplary embodiment the skin cholesterol may be estimated based on the measurement of reflected or transmitted light at wavelengths λ₁ and λ₂.

In other embodiments, the amount of skin cholesterol 126 present in the tissue sample may be determined by comparing the absolute or relative degree of differential absorption at λ₁ and λ₂ with a reference measurement 130. The reference measurement 130 may be empirically determined and may be selected or modified to account for patient specific factors, such as age, gender, weight, body mass index, diabetes, and/or other patient demographic or patient history factors. Such an implementation may, in one embodiment, compare an absolute or scaled value derived from the differential absorption of light at λ₁ and λ₂ with empirically derived values stored in a look up table such that, based upon the comparison, a skin cholesterol measurement 126 is determined form the look up table. Alternatively, the absolute or scaled value derived from the differential absorption of light at λ₁ and λ₂ may be input into one or more empirically or theoretically derived equations or formulas to solve for the skin cholesterol (or other sterol) measurement 126. In other embodiments, the absolute or scaled value derived from the differential absorption of light at λ₁ and λ₂ may be analyzed using at least one of a multivariate analysis or principal component analysis to derive the skin cholesterol measurement 126.

The skin cholesterol measurement 126 (or other sterol measurement in alternative embodiments) can be used by a physician or other caregiver in treating the patient. For example, based upon the skin cholesterol measurement 126, a physician may select one of a number of possible treatment options for a patient. For instance, based upon the measurement 126 a physician may prescribe one or more of a medication, a diet, and/or an exercise regime for the patient. Likewise, the physician may choose to establish a time table for follow up examinations based upon the skin cholesterol measurement 126.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims. Indeed, the present techniques may not only be applied to measurement of skin cholesterol, but also to other sterols or compositions susceptible to deposit in the skin.

What is claimed is:

1. A method for measuring accumulated cholesterol in tissue, comprising:
emitting light of two or more wavelengths toward a skin sample such that the light is differentially absorbed by plated or platable cholesterol in the skin sample;
detecting the differentially absorbed light; and
determining the amount of plated or platable cholesterol in the skin sample based on the differential absorption.

2. The method of claim 1, wherein the two or more wavelengths comprise at least one wavelength between about 1,200 nm to about 2,400 nm.

3. The method of claim 1, wherein the two or more wavelengths comprises at least one wavelength between about 1,462 nm to about 1,482 nm, between about 1,703 nm to about 1,723 nm, between about 1,740 nm to about 1,760 nm, between about 2,066 nm to about 2,086 nm, or between about 2,300 nm to about 2,320 nm.

4. The method of claim 1, wherein the skin sample is collected on an adhesive substrate.

5. The method of claim 1, wherein the skin sample is an in vivo skin sample.

6. The method of claim 1, wherein determining the amount of plated or platable cholesterol comprises comparing the differential absorption of the light to a reference measure.

7. The method of claim 1, wherein determining the amount of plated or platable cholesterol comprises solving a mathematical and/or statistical formula using the differential absorption of the light.

8. The method of claim 1, wherein determining the amount of plated or platable cholesterol comprises using at least one of multivariate analysis or principal component analysis.

9. A system for measuring accumulated cholesterol in tissue comprising:
one or more light emitters each configured to emit light at one or more wavelengths such that the combined one or more light emitters emit light at two or more wavelengths;
one or more light detectors configured to detect the light; and
a processor configured to receive the detected light and to determine an amount of plated or platable cholesterol in the skin based on differential absorption of the light.

10. The system of claim 9) comprising a sensor upon which the one or more light emitters and the one or more light detectors are disposed.

11. The system of claim 9, wherein the two or more wavelengths comprise at least one wavelength between about 1,200 nm to about 2,400 nm.

12. The system of claim 9, wherein the two or more wavelengths comprise at least one wavelength between about 1,462 nm to about 1,482 nm, between about 1,703 nm to about 1,723 nm, between about 1,740 nm to about 1,760 nm, between about 2,066 nm to about 2,086 nm, or between about 2,300 nm to about 2,320 nm.

13. The system of claim 9, wherein the processor is configured to determine the amount of plated or platable cholesterol by comparing the differential absorption of the light to a reference measure.

14. The system of claim 9, wherein the processor is configured to determine the amount of plated or platable cholesterol by solving a mathematical and/or statistical formula using the differential absorption of the light.

15. The system of claim 9, wherein the one or more light emitters comprise a broad spectrum emitter configured to simultaneously emit at each of the two or more wavelengths.

16. The system of claim 9, wherein the one or more light emitters comprise at least one of light emitting diodes or fiber optic waveguides.

17. A system for measuring a sterol deposited in the skin comprising:
one or more light emitters each configured to emit light at one or more wavelengths such that the combined one or more light emitters emit light at two or more wavelengths;
one or more light detectors configured to detect the light; and
a processor configured to receive the detected light and to determine an amount of a sterol deposited in the skin based on differential absorption of the light;
one or more optically transmissive structures in optical communication with at least some of the one or more light emitters and/or the one or more light detectors, wherein the one or more optically transmissive structures are configured to penetrate the surface of an in vivo skin sample.

18. The system of claim 17, comprising a sensor upon which the one or more optically transmissive structures are disposed.

19. The system of claim 17, wherein the one or more optically transmissive structures comprise fiber optic strands.
